(19) United States
(12) Patent Application Publication
(10) Pub. No.: US 2019/0336226 A1
(43) Pub. Date: Nov. 7, 2019

(54) TRAINING SYSTEM FOR ROBOT-ASSISTED LAPAROSCOPIC SURGERY

(71) Applicant: The Board of Regents of the University of Oklahoma, Norman, OK (US)

(72) Inventor: Alexander R. Raines, Oklahoma City, OK (US)

(21) Appl. No.: 16/401,965

(22) Filed: May 2, 2019

Related U.S. Application Data
(60) Provisional application No. 62/666,190, filed on May 3, 2018.
(60) Provisional application No. 62/772,354, filed on Nov. 28, 2018.

(51) Int. Cl. A61B 34/00; A61B 34/35; G09B 23/28

(52) U.S. Cl. CPC A61B 34/25; A61B 34/35; A61B 90/361; A61B 34/76; G09B 23/285

(57) ABSTRACT

A training system for robot-assisted laparoscopic surgery is described. The training system includes a surgeon console in operative communication with a medical surgical robot, comprising (1) a pair of hand-operated controllers for manipulation by a surgeon to control the medical surgical robot, and (2) at least one hand motion sensing device trained on the hand-operated controllers for generating information indicative of hand motions of the surgeon made while manipulating the pair of hand-operated controllers. The training system includes a procedural monitor configured to display images of movements of the surgical instruments by the medical surgical robot at an intracorporeal surgical site, and a hand motion monitor in operative communication with the hand motion sensing device and configured to display images of hand motions of the surgeon as detected by the hand motion sensing device during manipulation of the pair of hand-operated controllers by the surgeon.

### INCORPORATION BY REFERENCE

The present patent application claims priority to the provisional patent applications identified by U.S. Ser. No. 62/772,354, filed on Nov. 28, 2018; and 62/666,190, filed on May 3, 2018, the entire content of both applications being hereby incorporated herein by reference.

### BACKGROUND

Robot-assisted laparoscopic surgery is a rapidly growing field within multiple surgical disciplines. Proper training of the surgeon who conducts surgery with the robot-assisted laparoscopic tools is critical to the safe and accurate use of the robotic tools. It is to satisfying this need that the present disclosure is directed.

### BRIEF DESCRIPTION OF THE DRAWINGS

Several embodiments of the present disclosure are hereby illustrated in the appended drawings. It is to be noted however, that the appended drawings only illustrate several embodiments and are therefore not intended to be considered limiting of the scope of the present disclosure.

FIG. 1 is a schematic depiction of an exemplary hand motion analyzer (HMA) system constructed in accordance with the present disclosure, constructed to include a medical robotic system having robotically-controlled surgical instruments, a surgeon/trainee console, and a procedural monitor system

FIG. 2 is a schematic depiction in cross-section of the surgeon/trainee console of FIG. 1.

### DETAILED DESCRIPTION

With the relatively new technology of robot-assisted laparoscopic surgery, in conjunction with traditional video-based surgery or as compared to open surgery, arises the question about how to appropriately train surgeons in the dexterities and abilities needed to use them in an efficient, precise, and safe manner. An educational challenge during the training of current robotic surgical technologies is the lack of the ability of the trainer to simultaneously observe intracorporeal, in situ, movements of the robotic tools and the actual hand-movements (hand motions) of the surgeon, at the surgeon console, which control the movements of the robotic tools within the body. To be effective, surgical training must simultaneously address teaching technique and skill development related to the operation itself and to the use of surgical technologies. The present disclosure describes a training system and method which addresses a solution to this issue and is referred to herein as the Hand Motion Analyzer (HMA) system.

Additionally, a high percentage of surgeons incur work-related musculoskeletal disorders but the effort to address such disorders by surgical ergonomics education is inadequate. A partial explanation for this deficiency is that there has been a lack of an evidence-based framework for such instruction (Epstein, et al, “The Current State of Surgical Ergonomics Education in U.S. Surgical Training,” Annals of Surgery, January, 2018). The HMA system of the present disclosure can be used to collect and store data that can subsequently be analyzed, e.g., using conventional techniques, to identify ergonomic/motion efficiencies of the surgeon both to improve teaching of surgical techniques and to improve the ergonomic efficiency of hand movements made during robotic surgery.

Before describing various embodiments of the present disclosure in more detail by way of exemplary description, examples, and results, it is to be understood as noted above that the present disclosure is not limited in application to the details of methods and apparatus as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and apparatus of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error. Further, in this detailed description, each numerical value (e.g., temperature or time) should be read once as modified by the term “about” (unless already expressly so modified), and then read again as not so modified unless otherwise indicated in context. As noted, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. The use of the term “about” may mean a range including ±10% of the subsequent number unless otherwise stated.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Features of any of the embodiments disclosed herein may be combined with features of any of the other embodiments disclosed herein to create a new embodiment.

The HMA system, in one non-limiting embodiment, includes a hand motion video camera with wireless (or wired) connectivity to a (hand motion) video camera monitor. The hand motion video camera is trained on hand-operated controllers of the medical robotic system so as to capture a sequence of hand motions of a surgeon (e.g., a trainee) so that the hand motions resulting in the intracorporeal movements of the medical robot system are visible simultaneously in real time (i.e., hand motions are available substantially when the video of the intracorporeal movements is available as feedback during the procedure) by viewing the hand motion video camera monitor, e.g., at a remote location. In one embodiment, the hand motion video camera monitor which shows an image of the hand movements as the trainee is operating the hand-operated controllers placed adjacent to a procedural monitor, i.e., a surgical video monitor (i.e., the monitor which shows the intracorporeal movements of the robotic tools), allowing for concurrent viewing. This is particularly valuable in a training setting, allowing the trainer to better instruct the trainee on both intracorporeal and console-side techniques simultaneously. With the hand motion video camera pointed at the surgeon's hands operating the hand-operated controllers while performing robotic surgery, the trainer is able to observe both the hand motion video camera monitor (watching the motion of the surgeon's hands in the console), and the surgical video monitor (watching the robotic arms and tool motions in the patient's body). In one non-limiting embodiment, the HMA includes one or more hand motion video camera attached to a Surgeon Console of the Intuitive DaVinci® Robotic Surgical System, wherein the one or more hand motion video cameras are pointed at the hand-operated controllers so as to capture a sequence of video of the hands of the trainee or user of the surgical system. Alternatively, the same system of hand motion cameras and hand motion video camera monitor can be applied to other video-based surgical technologies as well. The images of hand motions seen via the hand motion video camera can be viewed on the hand motion video monitor which is near the surgeon/trainee, or can be observed by an observer or trainer who is substantially remote to the site of the robotic procedure. The hand motion video images can be viewed simultaneously by a trainer and one or more observers via the hand motion video camera monitor(s) at remote locations.

In one embodiment of the HMA system, the hand motion video camera is a wide-angle camera to enable better visualization of both hands simultaneously. A wide-angle camera has a field of view with a width greater than a height. In some examples, the width of the field of view is between 1.4-2.0 times the height of the field of view. In one embodiment, the hand motion video monitor can be separate from the monitor of the Surgeon Console. In another embodiment, the hand motion video camera(s) is integral to the Surgeon Console (i.e., is not a separately attached camera and the hand motion images are fed directly into the procedural monitor (which shows the tool in corporeal movements) in a picture-in-picture format, enabling the trainer or observer to more easily view the hand motions and tool movements simultaneously. As noted above, the HMA system described herein can be applied to other video-based surgical technologies/techniques aside from the Intuitive DaVinci® Robotic Surgical System.

The HMA is in important intraoperative adjunct to other already established pre-operative training options. Currently there are well established training modules which simulate both basic surgical skills and specific operative procedures. There are five virtual reality simulators currently available for robotic training. These include the Robotic Surgical Simulator (RoSS™; Simulated Surgical Systems, Buffalo, N.Y.); dV-Trainer™ (Mimic Technologies, Inc., Seattle, Wash.); SEP Robot™ (SimSurgery™, Norway); the Da Vinci® Skills Simulator (Intuitive Surgical, Sunnyvale, Calif.), and the Robotix Mentor™ (3D systems, formerly Symbionix, Israel). All of these simulators have been evaluated to have face validity (looks like what it simulates), content validity (accurately simulates the test condition) and construct validity (can differentiate between novice and expert except RoSS™).

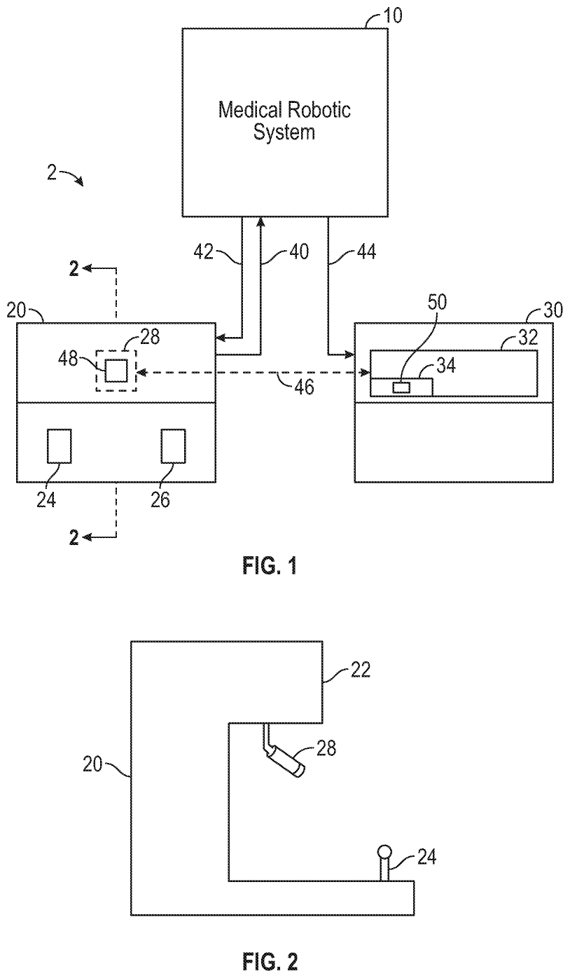

In one non-limiting embodiment, schematically represented in FIGS. 1 and 2, an HMA system 2, constructed in accordance with the present disclosure, comprises a medical robotic system 10, a surgeon console 20, and a procedural monitor system 30. The medical robotic system 10 comprises robotically-controlled surgical instruments under the control of a surgeon or trainee (via output 40 and input 42) that sits or stands at the surgeon console 20. Such medical robotic systems 10 are well known in the art (e.g., see U.S. Pat. Nos. 9,271,798 and 9,402,690). The surgeon console 20 comprises a viewing monitor 22, a pair of hand-operated controllers 24 and 26, and at least one hand motion sensing device 28 (which may be a video camera with a glass lens or a CCD-type digital camera or any other suitable video camera or visual imaging device) which is positioned above, below, and/or adjacent the hand-operated controllers 24 and 26 such that the movements and motions of the operator's hands are visible to the hand motion sensing device 28. In other words, hand motion sensing device 28 has a field of view encompassing the hand-operated controllers 24 and 26. The procedural monitor system 30 comprises a procedural monitor 32 which receives video signals from the medical robotic system 10 via input 44, and a hand motion monitor 34 (FIG. 2) which communicates with the hand motion sensing device 28 via a communication link 46. The hand motion monitor 34 can be a hand motion video camera monitor, in some embodiments. The communication link 46 can be a wired communication link such as an Ethernet cable (coaxial, twisted pair or otherwise) or a wireless communication link. Any suitable protocol can be used to communicate between the hand motion sensing device 28 and the hand motion monitor 34 via the communication link 46. For example, the communication link 28 can utilize unidirectional communication such as push technology to provide information from the hand motion sensing device 28 to the hand motion monitor 34, or bidirectional communication in which the hand motion monitor 34 provides control signals to the hand motion sensing device 28. Exemplary wireless protocols that can be used include protocols based upon the IEEE 802.11 suite of standards. These protocols are known in the art as “Wi-Fi” and use radio frequencies to extend wired Ethernet based local area networks to Wi-Fi enabled devices, as well as to permit and establish communication directly between Wi-Fi enabled devices. Exemplary protocols include IEEE 802.11a, 802.11b, 802.11g, 802.11-2007, 802.11n, 802.11-2012, 802.11ac, 802.11ad, 802.11af, 802.11-2016, 802.11ah, 802.11ai, 802.11aj, 802.11aq, 802.11ax, and 802.11ay. Other wireless protocols can also be used, such as a family of protocols known in the art as “Bluetooth”. Bluetooth is a wireless technology standard for exchanging data between fixed devices to other fixed devices; between mobile devices; and between fixed and mobile devices. Bluetooth uses short-wavelength UHF radio waves from 2.400 to 2.485 GHz. In one embodiment, the communication link 46 conforms to the requirements of the Internet. In this embodiment, the hand motion sensing device 28 can be an Internet Protocol camera (also known in the art as an IP camera). The hand motion monitor 34 can be a two-dimensional display device constructed of any suitable technology, such as Light Emitting Diode technology, Liquid Crystal Display technology, or the like. As discussed above, the communication link 46 enables wired or wireless communication between the hand motion sensing device 28 and the hand motion monitor 34. In one embodiment, the hand motion sensing device 28 includes a first transceiver 48, and the hand motion monitor 34 includes a second transceiver 50 communicating with the first transceiver 48 via the communication link 46, as discussed above.

The hand motion sensing device 28 may include a video camera that provides captured images to the hand motion monitor 34. The images may also be created by a computer system based upon captured images, depth maps or other information. In other embodiments, the hand motion sensing device 28 may capture information indicative of the location and/or movement of the trainee's hands on the hand-operated controllers 24 and 26. The information may be captured using image-triangulation techniques, medium reflectance techniques (LIDAR, radar, sonar or the like), or hand position sensing techniques utilized in natural user interfaces. Some natural user interfaces function by having an emitter project a medium onto a user's body (e.g., hands or wrists), a camera capture a representation of the medium as projected onto the body, and then interpret the representation of the medium to determine the three dimensional position and orientation of the body. Techniques for making and using a natural user interface are disclosed in U.S. Pat. No. 9,529,513, the entire content of which is hereby incorporated herein by reference.

The trainer sits or stands adjacent to the procedural monitor system 30 and by observing the procedural monitor 32 is able to view the movements of the surgical instruments at the surgical site within the surgical subject, and by observing the hand motion monitor 34 is able to view the movements of the surgeon's or trainee's hands as such hands manipulate the pair of hand-operated controllers 24 and 26. The hand motion monitor 34 may be separate from the procedural monitor 32, or may simply be a picture-in-picture image incorporated within the image of the procedural monitor 32, in a manner known in the art. In such a configuration, the trainer is able to view, almost simultaneously, both the movements of the surgical instruments at the surgical site and the movements of the surgeon's or trainee's hands and is therefore able to give the surgeon or trainee immediate instructions in real-time, for example if the hand movements of the surgeon/trainee require correction.

In one non-limiting embodiment, the HMA system 2 comprises (1) an Intuitive Surgical DaVinci® Robotic Surgical System, (2) one or more hand motion sensing device 28 with wireless connectivity attached to or positioned on or within the DaVinci® Robotic Surgical System, and directed toward the hand-operated controllers 24 and 26 to capture video of the surgeon's hands operating the hand-operated controllers 24 and 26, (3) a wireless hand motion monitor 34 (located at a convenient position where the trainer can concurrently view the intracorporeal procedure as well as the hand movements of the surgeon on the Surgeon Console). Examples of the DaVinci® Robotic Surgical System are shown in U.S. Pat. Nos. 9,271,798 and 9,402,690, each of which is hereby expressly incorporated herein by reference in its entirety.

The hand-operated controllers 24 and 26 can be any device that translates hand motion into operation commands for guiding the robotic surgery. Exemplary implementations of the hand-operated controllers 24 and 26, may include, but are not limited to, joysticks, buttons, natural interaction controllers, track ball(s) and the like.

During training, and once the surgeon/trainee has cleared the respective requirements to sit at the console, one or more hand motion sensing device 28 may be placed on the console (or in any other position) or attached thereto, or incorporated therein, in an orientation that is trained on the hand-operated controllers 24 and 26, and thus surgeon's hands while performing surgery. The hand motion video camera monitor 34 is set at an appropriate location near the procedural monitor 32, so the attending physician/trainer can easily view both the surgery and the surgeon's/trainee's hands in real time to deliver feedback to the surgeon/trainee when needed, as explained elsewhere herein.

When using the HMA system 2 described herein, for example during open and laparoscopic surgery, the trainer has wide view of both the operative field and the trainee hand motion, allowing the trainer to provide real time feedback to all aspects of the operation (e.g., surgical steps, correcting movements, instrument handling, and improvements in economy of motion). When the trainer is located remotely from the hand-operated controllers 24 and 26, the HMA system 2 may also be provided with a communication system (not shown) permitting verbal and/or video communication between the surgeon/trainee and the trainer so that real-time feedback can be presented from the trainer to the trainee. Prior to the presently disclosed HMA system 2, with robotic surgery, simultaneous visualization of all aspects of the operation/education has proven to be a challenge. The HMA system 2 provides a solution to such challenges, improving the effectiveness of surgical education in such settings.

### SUMMARY

Video-based surgeries are usually displayed on the procedural monitor 32; the intracorporeal (within the body) procedures are on display for both the surgeon and those observing within the operating room or remotely. The conventional setup for the Intuitive DaVinci® Robotic Surgical System does not readily allow for the simultaneous observation of both the intracorporeal ongoings and the actual hand movements of the surgeon using the hand controls on the Surgeon Console. Providing the hand motion sensing device 28 to record the hand operations manipulating the hand-operated controllers 24 and 26, and placing the remote hand motion monitor 34 of the HMA system 2 next to the procedural monitor 32 (or as a picture-in-picture system in the procedural monitor 32) showing the intracorporeal portions of the surgical procedure allows for simultaneous viewing of both. This is particularly important in a training setting, allowing the trainer to better instruct the trainee on both intracorporeal and console-side techniques simultaneously.

While the present disclosure has been described in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the presently disclosed systems, components, and methods. Changes may be made in various aspects of the systems, components, methods described herein without departing from the spirit and scope of the present disclosure.

What is claimed is:

1. A training system for robot-assisted laparoscopic surgery, comprising:
a medical surgical robot comprising surgical instruments for laparoscopic surgery;
a surgeon console in operative communication with the medical surgical robot, comprising (1) a pair of hand-operated controllers for manipulation by a surgeon to control the medical surgical robot, and (2) at least one hand motion sensing device trained on the hand-operated controllers for generating information indicative of hand motions of the surgeon made while manipulating the pair of hand-operated controllers; and
a procedural monitor system in operative communication with the surgeon console, comprising (1) a procedural monitor configured to display images of movements of the surgical instruments by the medical surgical robot at an intracorporeal surgical site, and (2) a hand motion monitor in operative communication with the hand motion sensing device and configured to display images of hand motions of the surgeon as detected by the hand motion sensing device during manipulation of the pair of hand-operated controllers by the surgeon.

2. The training system of claim 1, wherein the hand motion sensing device includes at least one video camera having a field of view encompassing the pair of hand-operated controllers, and wherein the information indicative of hand motions include images.

3. The training system of claim 2, wherein the images of hand motions include captured images.

4. A training system for robot-assisted laparoscopic surgery, comprising:
a medical surgical robot comprising surgical instruments for laparoscopic surgery;
a surgeon console in operative communication with the medical surgical robot, comprising (1) a pair of hand-operated controllers for manipulation by a surgeon to control the medical surgical robot, and (2) at least one hand motion sensing device trained on the hand-operated controllers for generating information indicative of hand motions of the surgeon made while manipulating the pair of hand-operated controllers; and
a procedural monitor system in operative communication with the surgeon console, comprising a procedural monitor configured to display (1) images of movements of the surgical instruments by the medical surgical robot at an intracorporeal surgical site, and (2) images of hand motions of the surgeon as detected by the hand motion sensing device during manipulation of the pair of hand-operated controllers by the surgeon.

5. The training system of claim 1, wherein the hand motion sensing device includes at least one video camera having a field of view encompassing the pair of hand-operated controllers, and wherein the information indicative of hand motions include images.

6. The training system of claim 5, wherein the images of hand motions include captured images.

7. A method of directing the operation of a medical surgical robot, comprising:
providing a medical surgical robotic system, comprising
(1) a medical surgical robot comprising surgical instruments for laparoscopic surgery,
(2) a surgeon console in operative communication with the medical surgical robot, comprising (a) a pair of hand-operated controllers for manipulation by a surgeon to control the medical surgical robot, and (b) at least one hand motion sensing device trained on the hand-operated controllers for generating information indicative of hand motions of the surgeon made while manipulating the pair of hand-operated controllers, and
(3) a procedural monitor system in operative communication with the surgeon console, comprising (a) a procedural monitor configured to display images of movements of the surgical instruments by the medical surgical robot at an intracorporeal surgical site, and (b) a hand motion monitor in operative communication with the hand motion sensing device and configured to display images of hand motions of the surgeon as detected by the hand motion sensing device during manipulation of the pair of hand-operated controllers by the surgeon;
observing the hand motions of the surgeon during manipulation of the medical surgical robot via the images displayed on the hand motion monitor; and
directing the surgeon to alter the manipulation of the pair of hand-operated controllers of the medical surgical robot when an alteration of the movement of the surgical instruments is desired.

8. The method of claim 7, wherein the hand motion sensing device includes at least one video camera having a field of view encompassing the pair of hand-operated controllers, and wherein the information indicative of hand motions include images.

9. The method of claim 8, wherein the images of hand motions include captured images.

10. A method of directing the operation of a medical surgical robot, comprising:
providing a medical surgical robotic system, comprising
(1) a medical surgical robot comprising surgical instruments for laparoscopic surgery,
(2) a surgeon console in operative communication with the medical surgical robot, comprising (a) a pair of hand-operated controllers for manipulation by a surgeon to control the medical surgical robot, and (b) at least one hand motion sensing device trained on the hand-operated controllers for generating information indicative of hand motions of the surgeon made while manipulating the pair of hand-operated controllers, and
(3) a procedural monitor system in operative communication with the surgeon console, comprising a procedural monitor configured to display (1) images of movements of the surgical instruments by the medical surgical robot at an intracorporeal surgical site, and (2) images of hand motions of the surgeon as detected by the hand motion sensing device during manipulation of the pair of hand-operated controllers by the surgeon;
observing the hand motions of the surgeon during manipulation of the medical surgical robot via the images displayed on the hand motion monitor; and
directing the surgeon to alter the manipulation of the pair of hand-operated controllers of the medical surgical robot when an alteration of the movement of the surgical instruments is desired.

11. The method of claim 10, wherein the hand motion sensing device includes at least one video camera having a field of view encompassing the pair of hand-operated controllers, and wherein the information indicative of hand motions include images.

12. The method of claim 11, wherein the images of hand motions include captured images.
